(19) United States
(12) Patent Application Publication
(10) Pub. No.: US 2020/0345755 A1
(43) Pub. Date: Nov. 5, 2020

(54) Treatment Regimens

(71) Applicant: NuCana plc, Edinburgh (GB)

(72) Inventor: Hugh Griffith, Edinburgh (GB)

(21) Appl. No.: 16/642,835

(22) PCT Filed: Aug. 30, 2018
(86) PCT No.: PCT/GB2018/052455

§ 371 Date: Feb. 27, 2020

(30) Foreign Application Priority Data
Aug. 30, 2017 (GB) 1713916.3

(51) Int. Cl. A61K 31/7068; G01N 33/50; G01N 33/574; A61P 35/00

(52) U.S. Cl. CPC A61K 31/7068; G01N 33/5011; A61K 9/0019; A61P 35/00; G01N 33/57496

(57) ABSTRACT

The invention relates to 5-fluoro-2′-deoxyuridine-5′-O-[1-naphthyl (benzoxy-L-alaninyl)] phosphate (NUC-3373), or a pharmaceutically acceptable salt thereof, for use in the treatment of cancer, in particular by intravenous infusion for a continuous period of up to 10 hours. The invention also relates to methods of treating cancer by administration of NUC-3373 to particular sub-groups of cancer patient. The invention further relates to methods for selecting a patient for treatment with NUC-3373.

The present invention relates to 5-fluoro-2′-deoxyuridine-5′-O-[1-naphthyl (benzoxy-L-alaninyl)] phosphate (NUC-3373), or a pharmaceutically acceptable salt thereof, for use in the treatment of cancer. The invention also relates to methods of treating cancer by administration of NUC-3373. The invention further relates to methods of assessing effectiveness of anti-cancer treatments. The invention further relates to patient groups that would particularly benefit from treatment with NUC-3373.

### BACKGROUND

### NUC-3373

Protides are masked phosphate derivatives of nucleosides. They have been shown to be particularly potent therapeutic agents in the fields of both antivirals and oncology. Protides appear to avoid many of the inherent and acquired resistance mechanisms which limit the utility of the parent nucleosides.

A ProTide adaptation of the nucleoside analogue, 5FUDR, 5-fluoro-2′-deoxyuridine-5′-O-[1-naphthyl (benzoxy-L-alaninyl)] phosphate (NUC-3373) 1 and a range of related compounds have shown activity in vitro against a range of cancer models, in many cases and in particular for NUC-3373 that activity was outstanding and far superior to the results obtained with 5-fluorouracil. The addition of the protide phosphoramidate moiety to the 5-fluorouracil/FUDR molecule confers the specific advantages of delivering the key activated form of the agent (FUDR monophosphate) into the tumour cells. Non clinical studies have demonstrated that NUC-3373 overcomes the key cancer cell resistance mechanisms associated with 5FU and its oral pro-drug capecitabine, generating high intracellular levels of the active FUDR monophosphate metabolite, resulting in a much greater inhibition of tumour cell growth. Furthermore, in formal dog toxicology studies, NUC-3373 is significantly better tolerated than 5FU (see WO2012/117246; McGuigan et al.; Phosphoramidate Pro Tides of the anticancer agent FUDR successfully deliver the preformed bioactive monophosphate in cells and confer advantage over the parent nucleoside; J. Med. Chem.; 2011, 54, 7247-7258; and Vande Voorde et al.; The cytostatic activity of NUC-3073, a phosphoramidate prodrug of 5-fluoro-2′-deoxyuridine, is independent of activation by thymidine kinase and insensitive to degradation by phosphorolytic enzymes; Biochem. Pharmacol.; 2011, 82, 441-452).

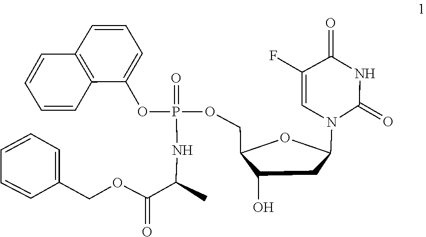 [SMILES: C[C@H](NP(=O)(OC[C@H]1O[C@@H](n2cc(F)c(=O)[nH]c2=O)C[C@H]1O)Oc1cccc2ccccc12)C(=O)OCc1ccccc1]

NUC-3373 1 is typically prepared as a mixture of two diastereoisomers, epimeric at the phosphate centre (the S-epimer and the R-epimer).

The therapeutic effect of 5-fluorouracil is through the formation of nucleotides that block normal nucleic acid formation. This is balanced by catabolism by dihydropyrimidine dehydrogenase (DPD) in the liver. Cancer patients that are dihydropyrimidine dehydrogenase deficient (DPD) or partially deficient are unable to degrade 5FU and other chemotherapeutic agents; most degradation occurs in the liver (DeLeve. Drug-Induced Liver Disease (Third Edition) 2013, pages 541-567). More than 85% of 5-fluorouracil is broken down by DPD; therefore, DPD activity is a major determinant of 5-fluorouracil is activity and toxicity. DPD enzyme activity follows a Gaussian distribution with up to six-fold inter-individual variation. In addition to the normal variation of DPD activity, there are also mutations in the DPYD gene that can lead to DPD deficiency, which occurs in less than 3% of the population. Low DPD levels and DPD deficiency reduce 5-fluorouracil clearance and can lead to severe or life-threatening toxicity.

In a clinical study it was observed that 55% of patients with a decreased DPD activity suffered from grade IV neutropenia compared with 13% of patients with a normal DPD activity (P=0.01). Furthermore, the onset of toxicity occurred, on average, twice as fast in patients with low DPD activity as compared with patients with a normal DPD activity (10.0+/−7.6 versus 19.1+/−15.3 days; P<0.05) (van Kullenburg et al., Clin Cancer res. 6(12):4705-12, 2000).

To date, 39 different mutations and polymorphisms have been identified in DPYD. The IVS14+1G>A mutation proved to be the most common one and was detected in 24-28% of all patients suffering from severe 5FU toxicity. Thus, a deficiency of DPD appears to be an important pharmacogenetic syndrome (van Kullenberg. Eur. J. Cancer. 40(7):939-50, 2004).

The reduced liver degradation of, for example, 5FU leads to prolonged exposure to the agent which can lead to profound toxicity, including mucositis, diarrhoea, granulocytopenia, neuropathy and death (Jin Ho Baek et al. Korean J International Medicine. 21:43-45, 2006). Conversely, patients that are not DPD deficient (e.g. normal DPD) are able to breakdown 5FU (and certain other chemotherapeutic agents) in the liver, however, 5FU is broken down to potentially toxic metabolites such as α-fluoro-β-alanine (FBAL) and dihydrofluorouracil (dhFU) presence of which is associated with hand-foot syndrome (also known as chemotherapy-induced acral erythema or palmar-plantar erythrodysesthesia, palmoplantar erythrodysesthesia) in 30-60% of patients (Kruger et al. Acta Oncologica 1-8, 2015; Chiara et al. Eur J Cancer. 33:967-969, 1997). Although not life threatening this can be debilitating.

There is therefore a need to identify treatments for cancer with a better safety profile (fewer side effects) than observed with existing therapies, such as 5FU, and capecitabine or Tegafur (prodrugs of 5FU), so that patients can either be treated with these treatments (e.g. agents) first line, or can be switched to these agents when the patient develops a side effect such as hand-foot syndrome, or is identified as a patient likely to develop such a side effect and/or less toxic side effects.

It is an aim of this invention to provide a therapy regime for treating cancer. It is also an aim of the present invention to provide methods for treating cancer patients who have DPD deficiency or partial DPD deficiency or who develop hand-foot syndrome. It is also an aim of this invention to provide methods for determining the effectiveness of a therapy for treating cancer. It is also an aim of this invention to provide methods for determining whether a subject's cancer is DPD deficient or partially deficient and providing a therapy for treating such a cancer.

It is an aim of this invention to provide a therapy that is more effective and/or less toxic/safer than existing treatments.

Certain embodiments of this invention satisfy some or all of the above aims.

### BRIEF SUMMARY OF THE DISCLOSURE

In a first aspect, the invention provides 5-fluoro-2′-deoxyuridine-5′-O-[1-naphthyl (benzoxy-L-alaninyl)] phosphate (NUC-3373), or a pharmaceutically acceptable salt thereof, for use in the treatment of cancer, wherein the treatment is by administration of NUC-3373 over a period of up to 10 hours, such as between 1 and 6 hours.

In a second aspect the invention provides a method of treating cancer, the method comprising administering 5-fluoro-2′-deoxyuridine-5′-O-[1-naphthyl (benzoxy-L-alaninyl)] phosphate (NUC-3373), or a pharmaceutically acceptable salt thereof, to a subject in need of such treatment over a period of up to 10 hours.

In a third aspect the invention provides the invention provides the use of 5-fluoro-2′-deoxyuridine-5-O-[1-naphthyl (benzoxy-L-alaninyl)] phosphate (NUC-3373), or a pharmaceutically acceptable salt thereof, in the manufacture of a medicament for use in the treatment of cancer, wherein the treatment is by administration of the medicament comprising NUC-3373 over a period of up to 10 hours.

In a fourth aspect, the invention provides a method of assessing effectiveness of an anti-cancer therapy, the method comprising:

assaying a sample of peripheral blood mononuclear cells (PBMCs) or cancer cells from a subject receiving an anti-cancer therapy to determine the level of intracellular deoxythymidine monophosphate (dTMP) within the PBMCs or cancer cells,

wherein a reduction in the level of intracellular dTMP within the PBMCs or cancer cells indicates that the anti-cancer therapy is effective.

In a fifth aspect, the invention provides a method of assessing effectiveness of an anti-cancer therapy, the method comprising:

assaying a sample of peripheral blood mononuclear cells (PBMCs) or cancer cells from a subject receiving an anti-cancer therapy to determine the level of intracellular thymidylate synthase (TS) within the PBMCs or cancer cells,

wherein a reduction in the level of intracellular TS within the PBMCs or cancer cells indicates that the anti-cancer therapy is effective.

On exposure to 5FU the levels of TS may increase and may contribute to resistance. Because treatment with NUC-3373 results in greater levels of the inhibitor FUDRMP it is predicted that treatment with NUC-3373 will be effective even in patients with increased levels of TS, that have typically arisen due to resistance mechanisms following treatment with agents such as 5FU.

In a sixth aspect, the invention provides a method of assessing effectiveness of an anti-cancer therapy, the method comprising:

assaying a sample of peripheral blood mononuclear cells (PBMCs) or cancer cells from a subject receiving an anti-cancer therapy to determine the level of intracellular deoxyuridine monophosphate (dUMP) within the PBMCs or cancer cells,

wherein an increase in the level of intracellular dUMP within the PBMCs or cancer cells indicates that the anti-cancer therapy is effective.

It will be appreciated that suitable cancer cells for use in the methods of the fourth to sixth aspects of the invention may be selected with reference to the subject's cancer. Peripheral blood mononuclear cells (PBMCs) are blood cells with nuclei, such as monocytes, lymphocytes, and macrophages.

In a seventh aspect, the invention provides 5-fluoro-2′-deoxyuridine-5′-O-[1-naphthyl (benzoxy-L-alaninyl)] phosphate (NUC-3373), or a pharmaceutically acceptable salt thereof, for use in the treatment of cancer by increasing intracellular levels of dUMP in treated cancer cells, and thereby causing cancer cell death. Without wishing to be bound by theory, in this scenario it may be that the cancer cell is killed due to a reduction in the efficacy of cellular DNA repair mechanism or due to cell stress.

In an eighth aspect, the invention provides 5-fluoro-2′-deoxyuridine-5′-O-[1-naphthyl (benzoxy-L-alaninyl)] phosphate (NUC-3373), or a pharmaceutically acceptable salt thereof, for use in the treatment of cancer in subjects that are deficient or partially deficient in dihydropyrimidine dehydrogenase (DPD).

In an embodiment, the eighth aspect of the invention provides 5-fluoro-2′-deoxyuridine-5′-O-[1-naphthyl (benzoxy-L-alaninyl)] phosphate (NUC-3373) or a pharmaceutically acceptable salt thereof, for use in treatment of a cancer in a subject that has been identified as being deficient or partially deficient in DPD.

In a ninth aspect, the invention provides a method of treating cancer, the method comprising administering 5-fluoro-2′-deoxyuridine-5′-O-[1-naphthyl (benzoxy-L-alaninyl)] phosphate (NUC-3373), or a pharmaceutically acceptable salt thereof, to a subject in need of such treatment wherein the subject is deficient or partially deficient in dihydropyrimidine dehydrogenase (DPD). The subject may be identified as deficient or partially deficient in DPD by any suitable means, including those disclosed herein.

NUC-3373 for use in accordance with any of the seventh to tenth aspects may be employed in accordance with any of the methods of treatment or medical uses, in their various aspects or embodiments, described herein.

In a tenth aspect, the invention provides a method of selecting a subject for treatment with 5-fluoro-2′-deoxyuridine-5′-O-[1-naphthyl (benzoxy-L-alaninyl)] phosphate (NUC-3373), or a pharmaceutically acceptable salt thereof, the method comprising determining whether the subject's liver cells, PBMCs or a cancer cell are deficient or partially deficient in DPD, wherein if the subject's liver cells, PBMCs or cancer cell are DPD deficient the subject is selected for treatment with 5-fluoro-2′-deoxyuridine-5′-O-[1-naphthyl (benzoxy-L-alaninyl)] phosphate (NUC-3373).

In an embodiment, the method of determining whether the subject's liver cells, PBMCs or a cancer cell are deficient or partially deficient in DPD is carried out by determining the amount or activity of DPD in a sample of PBMCs or cancer cells from the subject as compared to a reference value, wherein if the subject's PBMCs or cancer cells are deficient or partially deficient in DPD, the subject is selected for treatment with 5-fluoro-2′-deoxyuridine-5′-O-[1-naphthyl (benzoxy-L-alaninyl)] phosphate (NUC-3373).

In an eleventh aspect, the invention provides a method for determining whether a patient suffering with cancer is likely to benefit from or be responsive to treatment with 5-fluoro-2′-deoxyuridine-5′-O-[1-naphthyl (benzoxy-L-alaninyl)] phosphate (NUC-3373), or a pharmaceutically acceptable salt thereof, the method comprising determining the amount of activity of DPD protein expressed in a cancer cell isolated from the patient, wherein if the patient's cancer cell expresses reduced amount or DPD relative to a reference value, the patient is likely to benefit from or be responsive to treatment with 5-fluoro-2′-deoxyuridine-5′-O-[1-naphthyl (benzoxy-L-alaninyl)] phosphate (NUC-3373), or a pharmaceutically acceptable salt thereof. In an embodiment, determination of whether a patient is deficient or partially deficient in DPD is carried out on a cancer cell previously isolated from the patient suffering with cancer.

In a twelfth aspect, the invention provides use of 5-fluoro-2′-deoxyuridine-5′-O-[1-naphthyl (benzoxy-L-alaninyl)] phosphate (NUC-3373) or a pharmaceutically acceptable salt thereof, in a method of treating a subject with cancer which is deficient or partially deficient in DPD.

In a thirteenth aspect, the invention provides use of 5-fluoro-2′-deoxyuridine-5′-O-[1-naphthyl (benzoxy-L-alaninyl)] phosphate (NUC-3373) or a pharmaceutically acceptable salt thereof, in the manufacture of a medicament for use in a method of treating a subject with cancer, the method comprising:
(i) determining whether the subject's cancer is deficient or partially deficient in DPD and
(ii) administering to a subject whose cancer has been identified as being deficient or partially deficient in DPD a pharmaceutically effective amount of 5-fluoro-2′-deoxyuridine-5′-O-[1-naphthyl (benzoxy-L-alaninyl)] phosphate (NUC-3373).

In a particular embodiment, the subject's cancer is determined to be deficient or partially deficient in DPD by testing a cancer cell containing sample previously isolated from the subject for DPD activity, the presence of DPD protein, or surrogate thereof. As used herein the term patient and subject are used interchangeably.

By way of example, a suitable surrogate may include mRNA, DPD degradation products, or the ratio of dihydrouracil to uracil ratio in plasma.

In a fourteenth aspect, the invention provides 5-fluoro-2′-deoxyuridine-5′-O-[1-naphthyl (benzoxy-L-alaninyl)] phosphate (NUC-3373) or a pharmaceutically acceptable salt thereof, for use in the treatment of cancer in a subject that is at risk of developing or develops (has developed) hand-foot syndrome when being treated for their cancer by an agent other than NUC-3373. In particular embodiments, the patient is at risk of developing or develops hand-foot syndrome when being treated with or following treatment with 5FU or capecitabine. In a further aspect there is provided 5-fluoro-2′-deoxyuridine-5′-O-[1-naphthyl (benzoxy-L-alaninyl)] phosphate (NUC-3373) or a pharmaceutically acceptable salt thereof, for use in the treatment of cancer in a subject that suffers from hand-foot syndrome. In a particular embodiment, the subject has developed hand-foot syndrome following treatment with a drug other than NUC-3373. In particular embodiments, the drug other than NUC-3373′ is a fluoropyrimidine such as 5FU, capecitabine or tegafur.

It is known that 30-60% of patients treated with 5FU develop skin anomalies, such as hand-foot syndrome. Such patients typically express normal levels of DPD. Such potentially debilitating side effects are likely due to the build-up of toxic byproducts such as α-fluoro-β-alanine (FBAL) and dihydrofluorouracil (dhFU) when treated with anti-cancer agents such as 5FU and capecitabine (a prodrug of 5FU). FBAL and dhFU, were only detected at very low levels or were undetectable, respectively, following NUC-3373 administration at the doses studied and so NUC-3373 treatment is not expected to result in the side effects associated with FBAL and dhFU, such as hand-foot syndrome. This means that patients that develop hand-foot syndrome (or are at risk of developing this syndrome) when being treated for their cancer, such as when treated with 5FU or capecitabine, would benefit from switching to NUC-3373 treatment.

In an embodiment, the fourteenth aspect of the invention provides 5-fluoro-2′-deoxyuridine-5′-O-[1-naphthyl (benzoxy-L-alaninyl)] phosphate (NUC-3373) or a pharmaceutically acceptable salt thereof, for use in the treatment of a cancer in a subject that has developed hand-foot syndrome following treatment with 5FU or capecitabine.

In a fifteenth aspect, the invention provides a method of treating cancer, the method comprising administering 5-fluoro-2′-deoxyuridine-5′-O-[1-naphthyl (benzoxy-L-alaninyl)] phosphate (NUC-3373), or a pharmaceutically acceptable salt thereof, to a cancer subject with hand-foot syndrome. In particular embodiments, the patient has developed hand-foot syndrome by virtue of being treated with 5FU or capecitabine. In a particular embodiment, the patient's cancer treatment is switched to NUC-3373.

In a sixteenth aspect, the invention provides use of 5-fluoro-2′-deoxyuridine-5′-O-[1-naphthyl (benzoxy-L-alaninyl)] phosphate (NUC-3373) or a pharmaceutically acceptable salt thereof, in the manufacture of a medicament for use in a method of treating a subject with cancer, the method comprising:
(i) determining whether the cancer subject has hand-foot syndrome and
(ii) administering to the cancer subject with hand-foot syndrome a pharmaceutically effective amount of 5-fluoro-2′-deoxyuridine-5′-O-[1-naphthyl (benzoxy-L-alaninyl)] phosphate (NUC-3373).

In a seventeenth aspect, the invention provides a method of selecting a subject with cancer for treatment with 5-fluoro-2′-deoxyuridine-5′-O-[1-naphthyl (benzoxy-L-alaninyl)] phosphate (NUC-3373), or a pharmaceutically acceptable salt thereof, the method comprising determining whether the subject has hand-foot syndrome, wherein if the subject has hand-foot syndrome, the subject is selected for treatment with 5-fluoro-2′-deoxyuridine-5′-O-[1-naphthyl (benzoxy-L-alaninyl)] phosphate (NUC-3373).

NUC-3373 for use in accordance with any of the twelfth to seventeenth aspects may be employed in accordance with any of the methods of treatment or medical uses, in their various aspects or embodiments, described herein.

In an eighteenth aspect, the invention provides a method for determining whether delivery of an active anti-cancer agent into a cell has been achieved, the method comprising:
assaying a sample of peripheral blood mononuclear cells (PBMCs) or cancer cells from a subject receiving an anti-cancer therapy to determine the level of intracellular deoxyuridine monophosphate (dUMP) within the PBMCs or cancer cells, wherein an increase in the level of intracellular dUMP within the PBMCs or cancer cells indicates that delivery of the active anti-cancer agent into the cell has been achieved. In a particular embodiment the active agent is NUC-3373.

### BRIEF DESCRIPTION OF THE FIGURES

Embodiments of the invention are further described hereinafter with reference to the accompanying drawings, in which:

FIG. 1 shows the Cₘₐₓ and AUC for NUC-3373 in the blood plasma.

FIG. 2 shows the Cₘₐₓ and AUC for intracellular FUDR monophosphate.

### DETAILED DESCRIPTION

The first to third aspects of the present invention are based upon the inventors' surprising finding that NUC-3373 is retained in the circulation after administration for a period that is much longer than for 5-fluorouracil (5FU), the “parent” compound from which NUC-3373 is derived. As explained further in the Examples, the inventors have found that NUC-3373 has a plasma half-life of approximately 9.4 hours, as compared to a half-life of only 8-14 minutes for 5FU. This difference means that therapeutically effective levels of NUC-3373 are maintained for much longer after administration of this agent than after continual 46-48 hour administration of 5FU. As a consequence, effective anti-cancer treatment with NUC-3373 can make use of administration periods of 5/10 hours, or less, such as 3 or 4 hours, and even as little as 1 or 2 hours. This is in marked contrast to current treatment protocols with 5FU, the current standard of care for the treatment of many cancers. Here, the shorter half-life of 5FU requires the agent to be optimally administered to a patient over long periods, in order to achieve therapeutic activity. Treatment via constant infusion of 5FU over a period of 46-48 hours is commonplace, highlighting the short half-life (8-14 minutes) of 5FU. Sometimes this infusion is preceded by administration of a bolus dose (short infusion) of 5FU. Constant infusion refers to the administration of a fluid to a blood vessel, usually over a prolonged period of time.

It will be appreciated that the ability of the medical uses and methods of treatment of the invention to achieve therapeutically effective levels of NUC-3373 in a subject using much shorter periods of infusion offer many notable advantages. One major benefit of the medical uses and methods of treatment of the invention lies in the decreased disruption and invasiveness suffered by the recipient. Prior treatments' need for prolonged infusion periods, in the region of 46-48 hours, significantly decrease the quality of life experienced by the recipient. It will be appreciated that a reduced administration period results in a reduction in the time spend in hospital, and thus a likelihood of an increase in quality of life experienced by the recipient. A reduced administration period also results in decreased healthcare costs.

The methods of assessing effectiveness of an anti-cancer therapy provided by the fourth fifth, and sixth aspects of the invention arise from the inventors' surprising finding that treatment with NUC-3373 is able to deplete dTMP and TS levels and/or activity in the cells of subjects to whom it is administered and because dUMP is not converted to dTMP, treatment with NUC-3373 leads to an increased accumulation of intracellular dUMP. This is a new finding, and one that provides not only an indication of the way in which NUC-3373 may be achieving its therapeutic effect, but also provides a way in which the effectiveness of treatment may be monitored. Depletion of intracellular dTMP or TS levels or activity in PBMCs is not observed in patients receiving 5FU. Once again, this provides an indication of the different, and increased, effectiveness of NUC-3373 as compared to its parent compound. TS catalyses the conversion of deoxyuridine monophosphate (dUMP) to deoxythymidine monophosphate (dTMP). dTMP is subsequently phosphorylated to produce dTTP, a vital precursor for DNA replication and repair (Wilson et al., Nat Rev Clin Oncol. 11(5):282-98, 2014). Therefore, a reduction in the level of intracellular TS or inhibition of TS results in increased levels of dUMP in the cell which results in DNA damage.

It is this identification of a new mode of action of NUC-3373 that underlies the medical use of the seventh aspect of the invention. Recognition of this new mode of action, in which treatment with NUC-3373 increases intracellular accumulation of dUMP in cancer cells, thus causing the cells' death, enables novel uses of this compound in new clinical applications. In an embodiment of the seventh aspect of the invention, NUC-3373 may act to kill cancer cells by increasing levels of dUMP in the cells which results in DNA damage. The DNA damage then causes death of the cells, thereby bringing about effective treatment of cancer.

The medical uses and methods of treatment of the eighth, ninth, twelfth and thirteenth aspects of the present invention, and the patient selection and determination methods of the tenth and eleventh aspects of the invention, all relate to cancers that are deficient or partially deficient in DPD. These aspects are based upon the inventors' surprising finding that the toxic byproducts of 5FU administration, α-fluoro-β-alanine (FBAL) and dihydrofluorouracil (dhFU), were only detected at very low levels or were undetectable following NUC-3373 administration at the doses studied. Such byproducts are produced by 5FU and also produced when capecitabine, a prodrug of 5FU are administered, at clinically relevant amounts (leading to side effects such as hand-foot syndrome).

The medical uses and methods of treatment of the fourteenth to seventeenth aspects of the invention, all relate to cancer patients who develop side effects, such as hand-foot syndrome, when on chemotherapeutic agents, such as 5FU or capecitabine, due to the build-up of toxic metabolites (e.g. dhFU, FBAL) whose presence is associated with hand-foot syndrome. Again, these aspects are based upon the inventors' surprising finding that the toxic byproducts of 5FU administration, α-fluoro-β-alanine (FBAL) and dihydrofluorouracil (dhFU), were only detected at very low levels or were undetectable following NUC-3373 administration at the doses studied.

Since DPD is involved in pyrimidine degradation, subjects that are DPD deficient or partially deficient are unable to metabolise 5FU or capecitabine.

Patients with DPD deficiency who are treated with 5FU or capecitabine are at significantly increased risk of developing severe (grade III/IV) and potentially fatal neutropenia, mucositis and diarrhoea. DPD deficiency effects 5% of the population and a further 3-5% of the population are partially DPD deficient. The reduction in the amount of these by products (e.g. FBAL and dhFU) in patients treated with NUC-3373 therefore would suggest that DPD deficient and partially deficient patients can be treated with NUC-3373 whereas they could not be treated with 5FU or capecitabine. Therefore, NUC-3373 provides a lower risk treatment option for DPD deficient and partially deficient patients over 5FU or capecitabine.

Patients treated with 5FU or capecitabine that are not deficient in DPD, often develop hand-foot syndrome due to a build-up of the toxic byproducts FBAL and dhFU. As these byproducts are barely detectable in patients treated with NUC-3373, it is likely that fewer, if any, patients treated with NUC-3373 will suffer from hand-foot syndrome, or any other side effect mediated by these byproducts. Indeed, in the clinical trial disclosed in the Examples, none of the 36 patients treated have, so far, developed hand-foot syndrome. As hand-foot syndrome arises in 40-60% of patients treated with 5FU, if clinically meaningful levels of these by products were produced following administration of NUC-3373, it would have been expected that some patients would have developed hand-foot syndrome. This means that NUC-3373 is likely to be the suitable treatment choice for cancer patients who suffer from or are likely to develop hand-foot syndrome, or other FBAL- or dhFU-mediated side effects. For example, a patient being treated with 5FU who develops hand-foot syndrome could be switched from 5FU to NUC-3373 treatment; similarly, patients that are predicted to develop hand-foot syndrome if treated with a particular chemotherapeutic agent (e.g. 5FU or capecitabine) could be treated with NUC-3373 rather than the agent that is likely to result in the development of hand-foot syndrome. Patients that are more likely to develop hand-foot syndrome may be identified based on detection of one or more suitable surrogate markers (e.g. biomarkers) that detect susceptibility to developing hand-foot syndrome when treated with a chemotherapeutic drug, such as 5FU.

Further details of the various aspects and embodiments of the invention are described below. Technical considerations described in relation to any of the aspects or embodiments of the invention should also be taken as applicable to the other aspects and embodiments described herein, unless the context require otherwise.

The Compound

The 5-fluoro-2′-deoxyuridine-5′-O-[1-naphthyl (benzoxy-L-alaninyl)] phosphate (NUC-3373) may be a mixture of phosphate diastereoisomers or it may be present as the (S)-epimer in substantially diastereomerically pure form or as the (R)-epimer in substantially diastereomerically pure form.

It may be that the NUC-3373 is not in the form of a salt. Preferably, the NUC-3373 is in the form of the free base.

‘Substantially diastereomerically pure’ is defined for the purposes of this invention as a diastereomeric purity of greater than about 90% (about in this context means +/−5%). If present as a substantially diastereoisomerically pure form, the NUC-3373 may have a diastereoisomeric purity of greater than 95%, 98%, 99%, or even 99.5%.

Cancer to be Treated

The cancer may be a cancer selected from: pancreatic cancer, breast cancer, ovarian cancer, bladder cancer, other urothelial cancers, gastrointestinal cancer (also known as cancer of the digestive tract), liver cancer, lung cancer, biliary cancer, prostate cancer, cholangiocarcinoma, renal cancer, neuroendocrine cancer, sarcoma, lymphoma, leukemia, cervical cancer, thymic cancer, a cancer of an unknown primary origin, mesothelioma, adrenal cancer, cancer of the uterus, cancer of the fallopian tube, peritoneal cancer, endometrial cancer, testicular cancer, head and neck cancer, the central nervous system cancer, basal cell carcinoma, Bowens disease, other skin cancers (such as malignant melanoma, merckel cell tumour and rare appendage tumours), ocular surface squamous neoplasia and germ cell tumours.

The cancer may be selected from the group consisting of: leukaemia, lymphoma, pancreatic cancer, prostate cancer, lung cancer, breast cancer, cervical cancer, head and neck cancer, ovarian cancer, and gastrointestinal cancers. The gastrointestinal cancer may be selected from the group consisting of: oesophageal cancer, gastric cancer, stomach cancer, bowel cancer, small intestine cancer, colon cancer, appendix mucinous, goblet cell carcinoid, liver cancer, biliary cancer, gallbladder cancer, anal cancer and rectal cancer.

The cancer may be relapsed. The cancer may be metastatic. The cancer may be previously untreated. The cancer may be refractory cancer that has previously been treated but has proven unresponsive to prior treatment. Alternatively, the cancer patient may be intolerant of a previous therapy, for example, may develop side effects that make the patient intolerant to further treatment with the agent being administered. An example of this is the development of hand-foot syndrome when receiving certain anti-cancer therapies, like 5FU and capecitabine or tegafur.

Suitably, treatment in accordance with the medical uses or methods of the invention may be provided as a first line cancer therapy (i.e. the first cancer therapy provided after diagnosis of the disease). Alternatively, it may be used as a second or further line cancer treatment. It may be used as a third line or further cancer treatment.

Treatment Regime

The first, second and third aspects of the invention all relate to treatments requiring the administration of NUC-3373 over a period of up to 10 hours. The skilled reader will appreciate that, except for where the context requires otherwise, any of the medical uses and methods of treatment of the invention described herein (thus, including those of the seventh, eighth, ninth, twelfth, and thirteenth aspects of the invention) may employ administration of NUC-3373 over a period of up to 10 hours.

Suitably the NUC-3373 is for use in the treatment of cancer, where treatment is by administration of NUC-3373 over a period of up to 9 hours, up to 8 hours, up to 7 hours, up to 6 hours, or by administration of NUC-3373 over a period of up to 5 hours.

The NUC-3373 may be for use in the treatment of cancer, where treatment is by administration of NUC-3373 over a period of up to 4.75 hours, up to 4.5 hours, up to 4 hours, up to 3.75 hours, up to 3.5 hours, up to 3.25 hours, up to 3 hours, up to 2.75 hours, up to 2.5 hours, up to 2.25 hours, up to 2.25 hours, up to 2 hours, up to 1.75 hours, up to 1.5 hours, up to 1.25 hours, up to 1 hour, up to 0.75 hours, up to 0.5 hours, or by administration over a period of up to 0.25 hours.

Suitably the NUC-3373 is for administration over a period of between 1 and 6 hours, between 1 and 5 hours, between 1 and 4 hours, between 1 and 3 hours, between 2 and 4 hours, between 3 and 6 hours, between 3 and 5 hours or of between 1 and 2 hours.

Similarly, in methods of treatment in accordance with the various aspects of the invention, the NUC-3373 is suitably administered over a period of up to 9 hours, up to 8 hours, up to 7 hours, up to 6 hours, or up to 5 hours for the treatment of cancer.

For example, the NUC-3373 may be administered over a period of up to 4.75 hours, up to 4.5 hours, up to 4 hours, up to 3.75 hours, up to 3.5 hours, up to 3.25 hours, up to 3 hours, up to 2.75 hours, up to 2.5 hours, up to 2.25 hours, up to 2.25 hours, up to 2 hours, up to 1.75 hours, up to 1.5 hours, up to 1.25 hours, up to 1 hour, up to 0.75 hours, or up to 0.5 hours for the treatment of cancer.

### NUC-3373 Administration

Preferably, the administration is by means of infusion but could also be by, or include, a bolus administration.

The NUC-3373 may be administered parenterally, e.g. for intravenously, subcutaneously or intramuscularly. Preferably, the NUC-3373 is administered intravenously, for example, via a central or peripheral line.

The NUC-3373 may be administered parenterally as an aqueous formulation which optionally also comprises a polar organic solvent, e.g. DMA together with a surfactant. In the case of parenteral (e.g. intravenous) administration, the formulation preferably also comprises a polar aprotic organic solvent, e.g. DMA.

The formulation may be for dilution by a predetermined amount shortly before administration, i.e. up to 48 hours (e.g. up to 24, 12 or 2 hours) before administration.

The formulation may also comprise one or more pharmaceutically acceptable solubilizers, e.g. a pharmaceutically acceptable non-ionic solubilizers. Solubilizers may also be called surfactants or emulsifiers. Illustrative solubilizers include polyethoxylated fatty acids and fatty acid esters and mixtures thereof. Suitable solubilizers may be or comprise polyethoxylated castor oil (e.g. that sold under the trade name Kolliphor® ELP); or may be or comprise polyethoxylated hydroxy-stearic acid (e.g. that sold under the trade names Solutol® or Kolliphor® HS15); or may be or comprise polyethoxylated (e.g. polyoxyethylene (20)) sorbitan monooleate, (e.g. that sold under the trade name Tween® 80).

In certain preferred embodiments, the formulation comprises more than one pharmaceutically acceptable solubilizer.

The formulation may also comprise an aqueous vehicle. The formulation may be ready to administer, in which case it will typically comprise an aqueous vehicle.

The formulation may be for parenteral, e.g. for intravenous, subcutaneous or intramuscular administration. Preferably, the formulation is for intravenous administration. The administration may be through a central vein or it may be through a peripheral vein.

The formulation may be a formulation described in WO2017/109491.

While NUC-3373 is preferably formulated for parenteral administration, in certain embodiments of the invention it may be administered orally or topically.

For the above-mentioned uses and methods of the invention the dosage administered will, of course, vary with the precise mode of administration, the treatment desired and the disorder indicated. Dosage levels, dose frequency, and treatment durations of compounds of the invention are expected to differ depending on the formulation and clinical indication, age, and co-morbid medical conditions of the patient. The size of the dose for therapeutic purposes of compounds of the invention will naturally vary according to the nature and severity of the conditions, the age and sex of the animal or patient and the route of administration, according to well known principles of medicine.

The NUC-3373 may be administered in a dose in the range of from 100 mg/m²to 4000 mg/m², such as from 100 mg/m²to 3000 mg/m². The NUC-3373 may be administered in a dose in the range of from 500 mg/m² to 2000 mg/m². The NUC-3373 may be administered in a dose in the range of from 2000 mg/m²to 4000 mg/m²

The NUC-3373 may be administered on day 1 of a 28 day cycle. It may be administered on days 1, 8, 15 and 22 of a 28 day cycle. It may be administered on days 1 and 15 of a 28 day cycle.

It will be appreciated that a “cycle” is a course of treatment (treatment cycle), typically that is interspersed with periods of rest (no treatment). The NUC-3373 may be administered as part of a 4, 5, 6, 7 or more series of cycles. A series of cycles refers to a number of sequential cycles, typically interspersed with a period or rest (treatment vacation).

### Methods of Assessing Effectiveness of Anti-Cancer Therapy

The fourth, fifth, and sixth aspects of the invention provide methods of assessing effectiveness of an anti-cancer therapy. The methods of the third, fourth and fifth aspects of the invention are of particular utility in assessing the effectiveness of anti-cancer therapies in subjects receiving anti-cancer treatment using NUC-3373. For example, the methods of the third, fourth and fifth aspects of the invention may be utilised in respect of a subject receiving treatment with NUC-3373 used in a medical use of the invention (for example a medical use of the first, third, seventh, eighth, or thirteenth aspects of the invention), or receiving NUC-3373 in a method of treatment in accordance with the invention (for example a method of treatment of the second, ninth or twelfth aspects of the invention).

Suitably, the level of intracellular dTMP, dUMP or TS within the PBMCs or cancer cells may be compared to a suitable control value. Merely by way of example, a suitable control value may be representative of an intracellular dTMP, dUMP or TS level selected from: cells of the same subject prior to receiving the anti-cancer therapy; cells from an individual not receiving cancer therapy; and cells of an individual receiving cancer therapy with an agent other than NUC-3373. The control cells may be PBMCs or corresponding cancer cells. The control PBMCs or cancer cells may be collected in the same manner as the PBMCs or cancer cells of the subject. Suitably, control values may be generated from historical averages. Suitably, control values are obtained by testing cells from the same patient before treatment starts (baseline levels).

As noted above, a reduction in intracellular dTMP or TS level in PBMCs or cancer cells as compared to a suitable control (e.g. baseline pre-treatment level) is indicative of effective treatment. In contrast, an increase in intracellular dUMP level in PBMCs or cancer cells is indicative of effective treatment. Suitably, the reduction of intracellular dTMP or TS level is a reduction of at least 25%. Indeed, the reduction of intracellular dTMP level may be a reduction of at least 50%, at least 55%, at least 60%, at least 65%, at least 70%, at least 75%, at least 80%, at least 85%, at least 90%, or at least 95%.

In a suitable embodiment, the reduction of intracellular dTMP or TS level is a substantially complete reduction of intracellular dTMP or TS. For the purposes of the present disclosure, a reduction of intracellular dTMP or TS level of at least 95%, at least 96%, at least 97%, at least 98%, or at least 99% may be considered a substantially complete reduction of intracellular dTMP or TS.

NUC-3373 works by inhibiting intracellular TS. In a suitable embodiment, the inhibition of intracellular TS level is substantial inhibition of intracellular TS function. For the purposes of the present disclosure, a inhibition of intracellular TS of at least 95%, at least 96%, at least 97%, at least 98%, or at least 99% may be considered a substantially complete inhibition of intracellular TS.

An increase in intracellular dUMP indicative of effective cancer treatment may be an increase of at least 25%. Suitably the increase may be an increase of at least 50%, at least 55%, at least 60%, at least 65%, at least 70%, at least 75%, at least 80%, at least 85%, at least 90%, or at least 95%. Indeed, a suitable increase in intracellular dUMP may be an increase of 100% or more.

Intracellular dTMP, TS or dUMP levels may be determined at a time approximately in the range from 1 to 6 hours after the subject has begun anti-cancer treatment. Suitably, intracellular dTMP, TS or dUMP levels may be determined at a time approximately in the range from 1 to 6 hours after administration of an anti-cancer agent to the subject.

It will be appreciated that for the purposes of the present invention, the term “approximately” when referring to the time point for determining a biomarker, means plus or minus 1 hour. Suitably, intracellular dTMP or TS levels may be determined at any time during a treatment cycle. It will be appreciated that the relevant time is the time of sampling, rather than the time when the assay is carried out. For example, if a sample is taken after 6 hours, but frozen or treated in another way to ensure that the amount of a biomarker remains at roughly the same level, and the sample is then assayed after 12 hours it is the 6 hour sampling time rather than the 12 hour assaying time which is relevant.

Intracellular dTMP, TS or dUMP levels may be determined by any suitable assay or method known to the skilled person. A suitable assay for the determination of dTMP may include ultra-performance liquid chromatography-tandem mass spectrometry (UPLC-MS), as disclosed in the Examples below, or high performance liquid chromatography mass spectrometry (HPLC/MS). The same technique may be used for the assessment of intracellular dUMP levels.

Suitable methods or assays to determine TS levels may include Western blot, immunoassays, amino acid assays, or SDS-PAGE. Detection of TS levels by Western blot is disclosed in the Examples below.

For example, other suitable methods for determining levels of dUMP would also be known to the skilled person.

In the event that a subject exhibits minimal or no reduction in intracellular dTMP or TS, or minimal or no increase in intracellular dUMP, a treating physician may increase or terminate the dose of NUC-3373 received by the subject. It will be appreciated therefore that levels of dTMP, TS or dUMP may be used to inform the appropriate treatment for an individual patient.

### Dihydropyrimidine Dehydrogenase (DPD) Deficiency

The eighth to thirteenth aspects of the invention all relate to medical uses and methods wherein a subject is deficient or partially deficient in DPD.

DPD deficiency is an autosomal recessive metabolic disorder in which there is absent or significantly decreased activity of DPD, an enzyme involved in the metabolism of uracil and thymine. The decrease in activity may result from reduced expression of DPD, or expression of DPD with reduced function. DPD deficiency may be manifest as full deficiency or partial deficiency.

Standard techniques of determining whether a cell, such as a liver cell, PBMC or cancer cell, is deficient in DPD are well known and the skilled person would have the relevant knowledge to undertake the appropriate tests. Examples of such tests include those set out below.

Enzymatic activity of DPD in subjects with suspected DPD deficiency can be determined by assaying DPD protein extracted from liver cells, PBMCs or cancer cells. DPD activity may also be assessed by assaying for a surrogate of DPD protein such as RNA extracted from liver cells, PBMCs or cancer cells. Measurement of DPD mRNA copy number may then be undertaken. Nucleic acids encoding DPD may also be assayed with reference to presence or absence of DPD gene amplification, or presence or absence of DPD mutations indicative of DPD, or activity of DPD in suitable cells (such as liver cells, PBMCs or cancer cells) extracted from the subject of interest.

The enzyme DPD is encoded by the DPYD gene in humans. It is known that there are more than 50 mutations in the DPYD gene identified in people with DPD deficiency (Diagnostic Molecular Pathology: A Guide to Applied Molecular Testing, 2017, Edited by: William B. Coleman and Gregory J. Tsongalis).

Merely by way of example, a subject may have a genetic mutation selected from IVS14+1G>A mutation in intron 14 coupled with exon 14 deletion (known as DPYD*2A), 496A>G in exon 6; 2846A>T in exon 22; and T1679G (DPYD*13) in exon 13. Genetic variants in the DPYD gene on chromosome 1p21.3 have also been shown to result in deficient DPD activity (Diagnostic Molecular Pathology: A Guide to Applied Molecular Testing, 2017, Edited by: William B. Coleman and Gregory J. Tsongalis).

In an embodiment of the fifth, sixth, seventh, eighth, ninth or tenth aspect of the invention, the subject has a mutation in the DPYD gene that results in deficiency or partial deficiency of DPD.

In another embodiment of the fifth, sixth, seventh, eighth, ninth or tenth aspect of the invention, the genetic mutation in a subject that results in has a mutation in deficiency or partial deficiency of DPD may be selected from: IVS14+1G>A mutation in intron 14 coupled with exon 14 deletion (known as DPYD*2A), 496A>G in exon 6; 2846A>T in exon 22; and T1679G (DPYD*13) in exon 13.In another embodiment of the fifth, sixth, seventh, eighth, ninth or tenth aspect of the invention a subject has a mutation in chromosome 1p21.3 of the DPYD gene that results in deficiency or partial deficiency of DPD.

Testing for the IVS14+1G>A DPYD variant (DPYD*2A) is available (Terrazzino,S. et.al, Pharmacogenomics. 2013 Aug;14(11):1255-72).

High-throughput genetic analysis using denaturing high-performance liquid chromatography (DHPLC) can be used, particularly if the subject is severely neutropenic.

A subject may also be characterised as having DPD deficiency, or partial DPD deficiency, if they exhibit clinical or physiological characteristics of such a deficiency.

Merely by way of example, the subject may have previously exhibited intolerance for 5FU or capecitabine. Such intolerance is a known clinical characteristic of subjects with DPD deficiency or partial deficiency. Thus, in a suitable embodiment of the fifth, sixth, seventh, eighth, ninth or tenth aspect of the invention the subject has previously exhibited intolerance for 5FU or capecitabine.

Alternatively, or additionally, the subject may have a family history of intolerance for 5FU or capecitabine. Thus, in a suitable embodiment, a patient selected for use of NUC-3373 according to the fifth, sixth, seventh, eighth, ninth or tenth aspect of the invention has a family history of intolerance for 5FU or capecitabine.

A known physiological characteristic of subjects with DPD deficiency or partial deficiency is a change in the ratio of dihydrouracil to uracil ratio in plasma. A reduction in this ratio is indicative of DPD deficiency or partial deficiency. Thus, in a suitable embodiment, a subject may be identified as having DPD deficiency or partial deficiency by analysis of a plasma sample to assess the ratio of dihydrouracil to uracil, and comparison of this ratio to suitable reference values

In a particular embodiment, the patient's cancer may be determined to be DPD deficient or partially deficient by testing a cancer cell or PBMC containing sample that was previously isolated from the patient, for the presence of DPD protein, activity of DPD protein, or surrogate thereof (e.g. mRNA). In a particular embodiment, the patient's cancer may be determined to be DPD deficient or partially deficient by testing a cancer cell or PBMC containing sample for the presence of genetic mutations indicative of DPD deficiency or partial deficiency.

In an embodiment of the first and second aspects of the invention, NUC-3373 may be administered over a period of up to 10 hours to subjects deficient or partially deficient in DPD. The subject may have been identified as deficient or partially deficient in DPD and selected for treatment on this basis. DPD deficiency or partial deficiency may have been identified by means of any of the methods disclosed here.

In another embodiment of the first and second aspects of the invention, NUC-3373 may be administered over a period of up to 10 hours to subjects that have previously exhibited intolerance for 5FU or capecitabine. Suitably, the subjects may have a family history of intolerance for 5FU or capecitabine. The subject may have been selected for treatment on the basis of their intolerance for 5FU or capecitabine.

A subject may be determined to have DPD deficiency or partial deficiency if their DPD expression or function is determined to be reduced by at least 10% (as compared to a suitable reference value) as assessed by any of the relevant tests set out herein. Suitably a subject with DPD deficiency or partial deficiency may have a reduction in DPD expression or function of at least 25%, at least 30%, least 40%, at least 50%, least 60%, at least 70%, least 80%, or at least 90% as compared to a suitable reference value. Suitably a subject with DPD deficiency may have substantially no DPD expression or function as assessed by any of the relevant tests set out herein.

### Hand-Foot Syndrome

The fourteenth to seventeenth aspects of the invention all relate to medical uses and methods wherein a cancer patient/subject has or is likely to develop hand-foot syndrome when treated with a chemotherapeutic agent such as 5FU or capecitabine.

Hand-foot syndrome develops in 30-60% of patients treated with 5FU and 5FU related fluoropyrimidines (Kruger et al. Acta Oncologica 1-8, 2015; Chiara et al. Eur J Cancer. 33:967-969, 1997). It arises due to the build up of the 5FU toxic metabolites dhFU and FBAL. Hand-foot syndrome is a potentially dose-limiting cutaneous toxicity. It is characterized by paresthesia in a sock-and-glove distribution, with varying degrees of pain, tingling, dryness, erythema, scaling, swelling, and vesiculation of the hands and feet. Painful red swelling of the hands and feet in a patient receiving chemotherapy is usually enough to make the diagnosis of H&F syndrome.

In an embodiment, a cancer patient with hand-foot syndrome is selected for and treated with NUC-3373. In an embodiment, a cancer patient being treated with 5FU or capecitabine that develops hand-foot syndrome is treated with NUC-3373; typically, this will mean switching the patent from 5FU or capecitabine to NUC-3373, but it may also involve combination treatment wherein the patient is administered NUC-3373 in addition to the 5FU.

Rather than wait to switch treatment to NUC-3373 from an agent (e.g. therapeutic drug like 5FU) that results in the patient developing hand-foot syndrome, it should be possible to use appropriate genetic analyses (e.g. DPD genetics) to predict whether any particular patient is likely to develop hand-foot syndrome if administered 5FU or another chemotherapeutic agent that is known to be associated with hand-foot syndrome. In a particular embodiment, the patient is identified as possessing hand-foot syndrome based on physical examination (e.g. painful red swelling of the hands and feet in a patient receiving chemotherapy)

In one embodiment, following the treatment switch to NUC-3373 the hand-foot symptoms diminish. In a particular embodiment, the symptoms diminish entirely,

### Samples

It will be appreciated that the methods of the fourth, fifth, sixth, tenth and eleventh aspects of the invention make use of a “sample”, such as PBMCs or cancer cells. As used herein, the term “sample” typically refers to a biological sample obtained or derived from a subject requiring or undergoing treatment for cancer.

In some embodiments, a biological sample consists of or comprises biological tissue or fluid. As set out previously, a suitable sample may comprise cancer cells, liver cells and/or PBMCs. In some embodiments, a biological sample may be or comprise blood; blood cells; plasma; bone marrow ascites; tissue or fine needle biopsy samples; cell-containing body fluids; free floating nucleic acids; cell free circulating tumour DNA; sputum; saliva; urine; cerebrospinal fluid, peritoneal fluid; pleural fluid; faeces; lymph; gynaecological fluids; skin swabs; vaginal swabs; oral swabs; nasal swabs; washings or lavages such as a ductal lavages or broncheoalveolar lavages; aspirates; scrapings; secretions, and/or excretions; and/or cells therefrom.

In some embodiments, a biological sample is or comprises cells obtained from an individual. In some embodiments, obtained cells are or include cells from a subject. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, ascites, faeces).

In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification (e.g. polymerase chain reaction) or reverse transcription of mRNA, isolation and/or purification of certain components.

A suitable sample may be selected on the basis of its ability to contain an analyte to be analysed, such as DPD protein (or a surrogate thereof), dTMP, dUMP, TS, dhFU, or FBAL.

In some embodiments, the sample maybe a liquid, solid, or mixed biological sample obtained from a subject having, or suspected of having, a DPD deficient cancer. Suitable tissue samples include cancer tissue samples including those that may be obtained by a biopsy or following surgical resection of the cancer, surrounding tissues, and/or distant tissues in which metastasis are known or are suspected.

The diagnostic/determining methods of the invention can be undertaken using a sample previously taken from the individual or patient. Such samples may be preserved by freezing or fixed and embedded in formalin-paraffin or other media. Alternatively, a fresh cancer cell containing sample may be obtained and used directly or frozen and tested later.

As noted above, the presence of DPD protein can be detected in the cells, including the cell nuclei, using any of a variety of techniques. In particular embodiments, the presence of DPD protein is detected using immunohistochemistry, immunofluorescence, Western blotting, capillary electrophoresis, flow-cytometry or ELISA. Furthermore, these methods can be employed using an antibody or digital barcoded antibody to DPD protein. A digital barcoded antibody is an antibody whereby DNA barcodes are attached to the antibody. Multiple barcoded antibodies can then be assayed in parallel and subsequently analysed by DNA sequencing (e.g. see Agasti et al. J Am Chem Soc. 134(45):18499-18502, 2012)

In general, the level of DPD can be assessed using any of a variety of methods. In many embodiments, the level of DPD expression is assessed by determining the level of an DPD gene product in a sample obtained from a tumour. DPD protein level can also be determined using a surrogate of DPD protein, such as for example mRNA encoding DPD. Optionally the mRNA is detected directly or measured after conversion to cDNA which may optionally be amplified (e.g. by reverse transcriptase PCR).

The skilled person will readily be able to determine suitable reference values with respect to which the amount of the appropriate target molecule (e.g. DPD) may be compared. Merely by way of example, expression of target molecule in cancerous tissue can be compared to expression of that same molecule in non-cancerous tissue, such as adjacent non-cancerous tissue. Expression can be assessed on a protein level for example by immunohistochemistry or on a DNA level for example by fluorescence in situ hybridization, or on a RNA level, for example by quantitative real-time PCR.

### Compounds, Dosages Formulations of the Invention

Throughout this specification, the term S-epimer or S-diastereoisomer refers to 5-fluoro-2′-deoxyuridine-5′-O-[1-naphthyl (benzoxy-L-alaninyl)]-(S)-phosphate. Likewise, throughout this specification, the term R-epimer or R-diastereoisomer refers to 5-fluoro-2′-deoxyuridine-5-O-[1-naphthyl (benzoxy-L-alaninyl)]-(R)-phosphate.

The compounds of the invention may be obtained, stored and/or administered in the form of a pharmaceutically acceptable salt. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate, hemioxalate and hemicalcium salts. In certain embodiments, particularly those that apply to the s-epimer, the compound is in the form of a HCl salt or a hemioxalate salt.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the invention, the pharmaceutical composition which is used to administer the compounds of the invention will preferably comprise from 0.05 to 99%w (per cent by weight) compounds of the invention, more preferably from 0.05 to 80%w compounds of the invention, still more preferably from 0.10 to 70%w compounds of the invention, and even more preferably from 0.10 to 50%w compounds of the invention, all percentages by weight being based on total composition.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For parenteral (e.g. intravenous) administration the compounds may be administered as a sterile aqueous or oily solution. The compounds of the invention are very lipophillic. Aqueous formulations will typically, therefore, also contain a pharmaceutically acceptable polar organic solvent.

The present invention also includes all pharmaceutically acceptable isotopically-labelled forms of NUC-3373 wherein one or more atoms are replaced by atoms having the same atomic number, but an atomic mass or mass number different from the atomic mass or mass number of the predominant isotope usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds can generally be prepared by conventional techniques known to those skilled in the art or by processes analogous to those described using an appropriate isotopically-labelled reagent in place of the non-labelled reagent previously employed.

### Combinations

The method of treatment or the compound for use in the treatment of cancer may involve, in addition to the NUC-3373, conventional surgery or radiotherapy or chemotherapy. Such chemotherapy may include the administration of one or more other active agents.

Thus, the, each or any one of the pharmaceutical formulations may comprise another active agent.

The one or more other active agents may be one or more of the following categories of anti-tumour agents:
(i) antiproliferative/antineoplastic drugs and combinations thereof, such as alkylating agents (for example cyclophosphamide, nitrogen mustard, bendamustin, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, pemetrexed, cytosine arabinoside, and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, mitoxantrone and camptothecin);
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as finasteride;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [Herceptin™], the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib and 6-acrylamido-N-(3-chloro-4-fluorophenyl)-7-(3-morpholinopropoxy)-quinazolin-4-amine (Cl 1033), erbB2 tyrosine kinase inhibitors such as lapatinib); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib , tipifarnib and lonafarnib), inhibitors of cell signalling through MEK and/or AKT kinases, c-kit inhibitors, abl kinase inhibitors, P13 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™); thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon a; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); toll-like receptor modulators for example TLR-7 or TLR-9 agonists; checkpoint inhibitors, such as anti-PD1, anti-PD-L1 and anti-CTLA monoclonal antibodies such as: nivolumab, pembrolizumab, pidilizumab, atezolizumab, durvalumab and avelumab;
(viii) cytotoxic agents for example fludaribine (fludara), cladribine, pentostatin (Nipent™); irinotecan and oxaliplatin;
(ix) steroids such as corticosteroids, including glucocorticoids and mineralocorticoids, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph;
(x) targeted therapies, for example Pl3Kd inhibitors, for example idelalisib and perifosine; or compounds that inhibit PD-1, PD-L1 and CAR T; and
(xi) agents that enhance the anti-cancer effect of chemotherapeutic drugs, e.g. leucovorin.

The one or more other active agents may also be antibiotics.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The invention will now be further described with reference to the following Examples and accompanying Figures.

### EXAMPLE 1

### Pharmacokinetic Analysis from the NuTide: 301 Study

NuTide: 301 is a Phase 1 dose escalation study in patients with advanced solid tumors. All patients have metastatic spread. To date, data has been generated from 21 out of 36 patients enrolled in the study, having a median age of 57 (range 20 to 77) and having had an average of three (range two to five) prior chemotherapy regimens. There have been 10 primary cancer types, the majority of which (57%) are colorectal cancer.

The patients received NUC-3373, administered as a 30-minute to 2 hour intravenous injection on days 1, 8, 15 and 22 of a 28-day cycle regimen. Patients could remain on study and receive treatment until disease progression or unacceptable toxicity occurs.

NUC-3373 is presented as a single dose intravenous injection in a clear vial containing 250 mg/ml NUC-3373 in a solution of dimethylacetamide (DMA) and normal saline in the ratio of 80:20. The product is a clear yellow solution, free from visible particles.

In the study all patients were treated with a 1:1 mixture of NUC-3373 S and R epimers.

The cohorts treated to date received 125 mg/m², 250 mg/m², 500 mg/m² or 750 mg/m² NUC-3373 per administration.

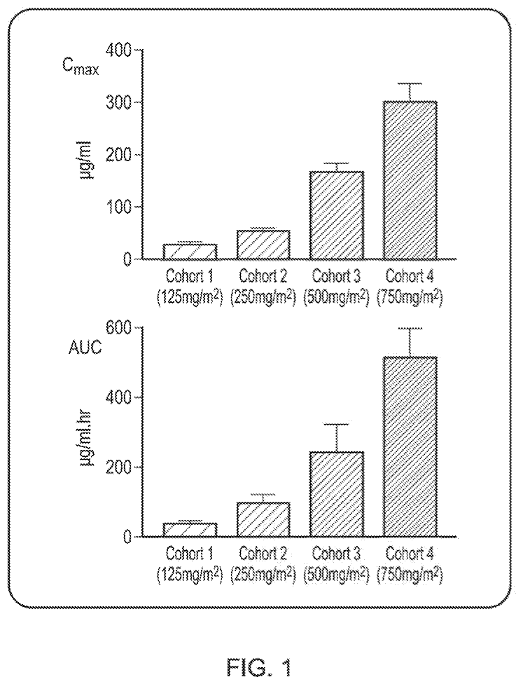

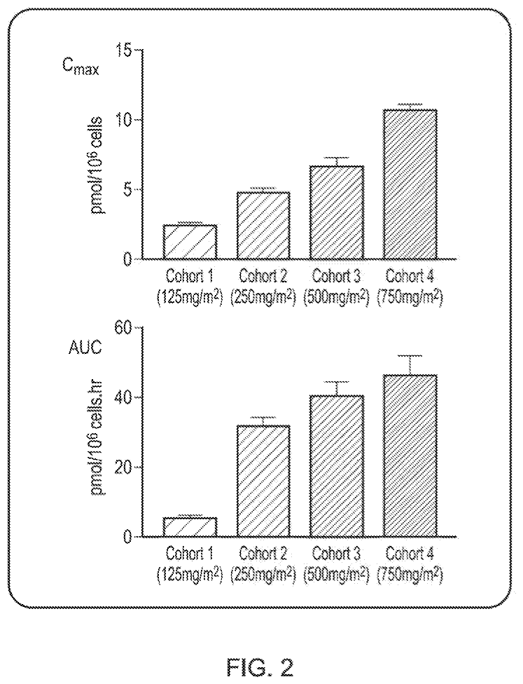

Pharmacokinetic analysis of blood samples of the patients was then carried out. The results are shown in FIGS. 1 and 2.

Blood samples were collected on days 1 and 15 of a treatment cycle:

The blood samples were taken at the following 12 time points listed below in Table 1.

TABLE 1
Sampling Schedule

| | |
|---|---|
| Sample 1 | T0 pre-dose (before the start of the infusion) |
| Sample 2 | T1 (immediately after drug has cleared the infusion line) |
| Sample 3 | T1 plus 15 minutes |
| Sample 4 | T1 plus 30 minutes |
| Sample 5 | T1 plus 45 minutes |
| Sample 6 | T1 plus 1 hour |
| Sample 7 | T1 plus 1.5 hours |
| Sample 8 | T1 plus 2 hours |
| Sample 9 | T1 plus 4 hours |
| Sample 10 | T1 plus 6 hours |
| Sample 11 | T1 plus 24 hours |
| Sample 12* | T1 plus 48 hours |

*Optional

Processing of Plasma Samples—4ml Lithium Heparin Vacutainer

The blood samples should arrive in the lab within 2 hours of collection. Centrifuge the 4 ml blood sample at 1,200g at 18° C. for 10 minutes. Using a sterile plastic pipette (pastette), remove the resulting plasma and transfer ˜1.0 ml plasma into each of 2 cryovials (2 ml).

Processing of PBMC Sample—8 ml CPT Tubes

The blood samples should arrive in the lab within 2 hours of collection. Collect 8 ml of blood into CPT tubes (blood tubes should be centrifuged within 2 hours of blood collection). Remix the blood sample immediately prior to centrifugation by gently inverting the tube 8 to 10 times. Set centrifuge so that the start and finish is set to a slow acceleration (without break). Centrifuge at 18° C. for 20 minutes at 1,500g. Remove sample from the centrifuge carefully. This will result in five layers: plasma (first); whitish cell (PBMC) layer [second]; polyester gel [third]; density solution [fourth] and, remaining granulocytes and RBCs [fifth]. Aspirate approximately half of the plasma without disturbing the cell layer. Collect the PBMC layer with a Pasteur Pipette and transfer to a 50 ml tube and add cold PBS (4° C.) to a final volume of 40 ml. Divide between two 50 ml tubes (20 ml per tube) and centrifuge for 5 minutes at 4° C. at 1,500g. Decant out the supernatant without disturbing the cell pellet and re-suspend the pellets in the residual buffer. Add 1 ml PBS to each pellet to gather up the cells and transfer one pellet to a 2 ml screw cap tube labelled ‘PD PBMCs’ and the other to a 2 ml screw cap tube labelled ‘PK PBMCs Pellet’. Centrifuge both tubes for 5 minutes at 4° C. at 1,500 g. Remove the supernatant with a pipette (p 1,000 μl). Please ensure that the supernatant is completely removed from the tube to ensure good quality sample. Add 200 μl of freezing media solution (provided, 4° C.) to the tube labelled ‘PD PBMCs’ and store at −80° C. until analysis.

Re-suspend the cell pellets in the tube labelled ‘PK PBMCs Pellet’ by adding 200 μl of 8−% Methanol (4° C.) and gently pipette up and down for 3 times. Vortex-mix for 30 seconds. Leave sample on ice for 15 minutes. Centrifuge at 4° C. for 5 minutes at 1,500 g. Using a micropipette, carefully transfer 180 μl of the supernatant to a 2 ml screw cap tube labelled ‘PK PBMC Supernatant’ without disturbing the cell pellet. Store both the PBMC pellet and PBMC supernatant at −80° C. until analysis for dTMP, TS, dUMP, FBAL and dhFU.

UPLC-MS for was used to detect dTMP Western blot was used to detect TS.

UPLC-MS/MS was used to detect dhFU and FBAL

FIG. 1 shows the Cₘₐₓ and AUC for NUC-3373 in the blood plasma for the cohorts treated to date. The half-life of NUC-3373 in plasma was 9.7 hours. In contrast, 5FU has a plasma half-life of 8-14 minutes. The toxic byproducts α-fluoro-β-alanine (FBAL) and dihydrofluorouracil (dhFU) were undetectable following NUC-3373 administration at the doses studied.

FIG. 2 shows the Cₘₐₓ and AUC for intracellular FUDR monophosphate for the cohorts treated to date. The half-life of FUDR monophosphate was found to be 14.9 hours. FUDR monophosphate was still detectable at 48 hours.

It is known that accumulation of dUMP within cells leads to DNA damage, and that this DNA damage is associated with cell death. Thus, the ability of NUC-3373 to promote accumulation of dUMP within cancer cells represents a previously unrecognized mode of action by which NUC-3373 is able to kill cancer cells, and thereby effectively treat cancer.

1. 5-fluoro-2′-deoxyuridine-5′-O-[1-naphthyl (benzoxy-L-alaninyl)] phosphate (NUC-3373), or a pharmaceutically acceptable salt thereof, for use in the treatment of cancer, wherein the treatment is by administration of NUC-3373 over a period of up to 10 hours.

2. NUC-3373 for use according to claim 1, wherein the treatment is by administration of NUC-3373 over a period of up to 5 hours.

3. NUC-3373 for use according to claim 1 or claim 2, wherein the treatment is by administration of NUC-3373 over a period of up to 2 hours.

4. NUC-3373 for use according to claim 1, wherein the treatment is by administration of NUC-3373 over a period of between 1 and 2 hours, 2 and 4 hours or 1 and 6 hours.

5. NUC-3373 for use according to any preceding claim, wherein the administration is by means of continuous infusion.

6. NUC-3373 for use according to claim 5, wherein the infusion is by intravenous infusion.

7. NUC-3373 for use according to any of the preceding claims, wherein the treatment is by administration of NUC-3373 by means of or includes a bolus administration.

8. NUC-3373 for use according to any preceding claim, wherein the cancer is selected from the group consisting of: pancreatic cancer, breast cancer, ovarian cancer, bladder cancer, other urothelial cancers, gastrointestinal cancer (also known as cancer of the digestive tract), liver cancer, lung cancer, biliary cancer, prostate cancer, cholangiocarcinoma, renal cancer, neuroendocrine cancer, sarcoma, lymphoma, leukemia, cervical cancer, thymic cancer, a cancer of an unknown primary origin, mesothelioma, adrenal cancer, cancer of the uterus, cancer of the fallopian tube, peritoneal cancer, endometrial cancer, testicular cancer, head and neck cancer, the central nervous system cancer, basal cell carcinoma, Bowens disease, other skin cancers (such as malignant melanoma, merckel cell tumour and rare appendage tumours), ocular surface squamous neoplasia and germ cell tumours.

9. NUC-3373 for use according to claim 8, wherein the cancer is a gastrointestinal cancer selected from the group consisting of: oesophageal cancer, gastric cancer, stomach cancer, bowel cancer, small intestine cancer, colon cancer, appendix mucinous, goblet cell carcinoid, liver cancer, biliary cancer, gallbladder cancer, anal cancer and rectal cancer.

10. NUC-3373 for use according to any preceding claim, wherein the patient with the cancer also suffers from hand-foot syndrome.

11. NUC-3373 for use according to claim 10, wherein the patient has developed hand-foot syndrome from a previous treatment regimen with a drug other than NUC-3373.

12. NUC-3373 for use according to claim 11, wherein the patient has developed hand-foot syndrome when being treated with 5FU, capecitabine or tegafur.

13. 5-fluoro-2′-deoxyuridine-5-O-[1-naphthyl (benzoxy-L-alaninyl)] phosphate (NUC-3373) or a pharmaceutically acceptable salt thereof, for use in the treatment of cancer in a subject that suffers from hand-foot syndrome.

14. NUC-3373 for use according to claim 14, wherein the subject has developed hand-foot syndrome following treatment with a fluoropyrimidine such as 5FU, capecitabine or tegafur.

15. NUC-3373 for use according to claim 14 or 15, wherein the cancer is selected from the group consisting of: pancreatic cancer, breast cancer, ovarian cancer, bladder cancer, other urothelial cancers, gastrointestinal cancer (also known as cancer of the digestive tract), liver cancer, lung cancer, biliary cancer, prostate cancer, cholangiocarcinoma, renal cancer, neuroendocrine cancer, sarcoma, lymphoma, leukemia, cervical cancer, thymic cancer, a cancer of an unknown primary origin, mesothelioma, adrenal cancer, cancer of the uterus, cancer of the fallopian tube, peritoneal cancer, endometrial cancer, testicular cancer, head and neck cancer, the central nervous system cancer, basal cell carcinoma, Bowens disease, other skin cancers (such as malignant melanoma, merckel cell tumour and rare appendage tumours), ocular surface squamous neoplasia and germ cell tumours.

16. NUC-3373 for use according to any of claims 14 to 16, wherein the cancer is gastrointestinal cancer and is selected from the group consisting of: oesophageal cancer, gastric cancer, stomach cancer, bowel cancer, small intestine cancer, colon cancer, appendix mucinous, goblet cell carcinoid, liver cancer, biliary cancer, gallbladder cancer, anal cancer and rectal cancer.

17. A method of selecting a subject with cancer for treatment with 5-fluoro-2′-deoxyuridine-5-O-[1-naphthyl (benzoxy-L-alaninyl)] phosphate (NUC-3373), or a pharmaceutically acceptable salt thereof, the method comprising determining whether the subject has hand-foot syndrome, wherein if the subject has hand-foot syndrome, the subject is selected for treatment with 5-fluoro-2′-deoxyuridine-5′-O-[1-naphthyl (benzoxy-L-alaninyl)] phosphate (NUC-3373).

18. The method according to claim 18, wherein the patient developed hand-foot syndrome whilst being treated with a fluoropyrimidine, such as 5FU or capecitabine.

19. 5-fluoro-2′-deoxyuridine-5-O-[1-naphthyl (benzoxy-L-alaninyl)] phosphate (NUC-3373), or a pharmaceutically acceptable salt thereof, for use in the treatment of cancer in subjects that are deficient or partially deficient in dihydropyrimidine dehydrogenase (DPD).

20. NUC-3373 for use according to claim 20, wherein the cancer is selected from the group consisting of: pancreatic cancer, breast cancer, ovarian cancer, bladder cancer, other urothelial cancers, gastrointestinal cancer (also known as cancer of the digestive tract), liver cancer, lung cancer, biliary cancer, prostate cancer, cholangiocarcinoma, renal cancer, neuroendocrine cancer, sarcoma, lymphoma, leukemia, cervical cancer, thymic cancer, a cancer of an unknown primary origin, mesothelioma, adrenal cancer, cancer of the uterus, cancer of the fallopian tube, peritoneal cancer, endometrial cancer, testicular cancer, head and neck cancer, the central nervous system cancer, basal cell carcinoma, Bowens disease, other skin cancers (such as malignant melanoma, merckel cell tumour and rare appendage tumours), ocular surface squamous neoplasia and germ cell tumours.

21. NUC-3373 for use according to claim 20 or 21, wherein the cancer is gastrointestinal cancer and is selected from the group consisting of: oesophageal cancer, gastric cancer, stomach cancer, bowel cancer, small intestine cancer, colon cancer, appendix mucinous, goblet cell carcinoid, liver cancer, biliary cancer, gallbladder cancer, anal cancer and rectal cancer.

22. NUC-3373 for use according to any one of claims 20 to 22, wherein the subject has a genetic mutation selected from IVS14+1G>A mutation in intron 14 coupled with exon 14 deletion (known as DPYD*2A), 496A>G in exon 6; 2846A>T in exon 22; and T1679G (DPYD*13) in exon 13.

23. NUC-3373 for use according to claim 23, wherein the subject has the IVS14+1G>A DPYD variant (DPYD*2A) mutation.

24. NUC-3373 for use according to any one of claims 20 to 22, wherein the subject has previously exhibited intolerance for 5FU or capecitabine or has a family history of intolerance for 5FU or capecitabine.

25. A method of assessing effectiveness of an anti-cancer therapy, the method comprising: assaying a sample of peripheral blood mononuclear cells (PBMCs) or cancer cells from a subject receiving an anti-cancer therapy to determine the level of intracellular deoxythymidine monophosphate (dTMP) within the PBMCs or cancer cells, wherein a reduction in the level of intracellular dTMP within the PBMCs or cancer cells indicates that the anti-cancer therapy is effective.

26. A method according to claim 26, wherein the subject is receiving anti-cancer treatment using NUC-3373.

27. A method according to claim 26 or claim 27, wherein the level of intracellular dTMP within the PBMCs or cancer cells is compared to a suitable control value.

28. A method according to any of claims 26 to 28, wherein the reduction is a reduction of at least 25%.

29. A method according to claim 29, wherein the reduction is substantially a complete reduction of intracellular dTMP.

30. A method of assessing effectiveness of an anti-cancer therapy, the method comprising: assaying a sample of peripheral blood mononuclear cells (PBMCs) or cancer cells from a subject receiving an anti-cancer therapy to determine the level of intracellular thymidylate synthase (TS) within the PBMCs or cancer cells, wherein a reduction in the level of intracellular TS within the PBMCs or cancer cells indicates that the anti-cancer therapy is effective.

31. A method according to claim 31, wherein the subject is receiving anti-cancer treatment using NUC-3373.

32. A method according to claim 31 or claim 32, wherein the level of intracellular TS within the PBMCs or cancer cells is compared to a suitable control value.

33. A method according to any of claims 31 to 33, wherein the reduction is a reduction of at least 25%.

34. A method according to claim 34 wherein the reduction is substantially a complete reduction of intracellular TS.
